(19) United States
(12) Patent Application Publication
(10) Pub. No.: US 2024/0168027 A1
(43) Pub. Date: May 23, 2024

(54) BIOMARKER FOR DIAGNOSIS, TREATMENT, AND PROGNOSIS FOR HEPATOCELLULAR CARCINOMA BONE METASTASIS AND APPLICATION THEREOF

(71) Applicants: SUN YAT-SEN UNIVERSITY CANCER CENTER, Guangzhou (CN); SUN YAT-SEN UNIVERSITY, Guangzhou (CN)

(72) Inventors: Jun LI, Guangzhou (CN); Libing Song, Guangzhou (CN); Shuxia ZHANG, Guangzhou (CN); Xinyi LIAO, Guangzhou (CN); Suwen CHEN, Guangzhou (CN); Wanying QIAN, Guangzhou (CN)

(21) Appl. No.: 18/325,792

(22) Filed: May 30, 2023

Related U.S. Application Data
(63) Continuation-in-part of application No. PCT/CN2022/135070, filed on Nov. 29, 2022.

(30) Foreign Application Priority Data
Apr. 26, 2022 (CN) 202210449059.3

(51) Int. Cl. G01N 33/574

(52) U.S. Cl. CPC G01N 33/5748

(57) ABSTRACT

The invention belongs to the technical field of biomedical testing, and particularly relates to a biomarker for diagnosis, treatment, and prognosis for hepatocellular carcinoma bone metastasis (HCC) and application thereof. The application of biomarker VAPA for preparing a reagent or a kit for diagnosis, treatment, and prognosis for HCC bone metastasis. The application of the reagent that detects VAPA expression levels in blood and tissues for preparing a reagent or kit for diagnosis, treatment, and prognosis for HCC bone metastasis. Compared with the diagnostic kits in the prior art that can only detect bone metastasis after it has occurred, the VAPA described in this invention offers a more specific and sensitive assay kit for the early prediction, diagnosis, disease progression assessment, treatment efficacy evaluation, drug guidance, and prognosis assessment of HCC bone metastasis. Additionally, the VAPA may also serve as a target for therapeutic interventions in bone metastasis.

### CROSS REFERENCE OF RELATED APPLICATIONS

This application is a continuation of PCT International Patent Application No. PCT/CN2022/135070, filed on 29 Nov. 2022, entitled “Biomarker for Diagnosis, Treatment, and Prognosis for Hepatocellular Carcinoma Bone Metastasis and Application thereof,” which claims foreign priority of Chinese Patent Applications No. 202210449059.3, filed 26 Apr. 2022 in the China National Intellectual Property Administration (CNIPA), the entire contents of which are hereby incorporated by reference in their entireties.

### FIELD OF THE INVENTION

The invention belongs to the technical field of biomedical testing, and particularly relates to a biomarker for diagnosis, treatment, and prognosis for hepatocellular carcinoma bone metastasis and application thereof.

### BACKGROUND OF THE INVENTION

Hepatocellular carcinoma (HCC) is one of the prevalent malignant tumors. With the advancements in diagnostic techniques and treatment methods for HCC, the overall survival of HCC patients has significantly increased. However, this also provides an extended period for the HCC metastatic cells to recur. Approximately 38.5% of HCC patients with distant metastasis exhibit bone metastasis (BM) at the time of initial diagnosis, and 11.7% of HCC patients undergoing curative resection will develop bone metastasis. The prognosis for HCC patients with bone metastasis is exceedingly poor, with a median survival time of merely 4.6 months. Additionally, most HCC patients with bone metastasis experience severe skeletal-related events, such as pathological fractures, spinal cord compression, severe pain, and neurological deficits, leading to a diminished quality of life. Nevertheless, due to the historically short survival period of HCC patients before the 20th century, where distant metastasis was not considered a clinical challenge, the underlying mechanisms of HCC bone metastasis remain unclear, and practical guidelines for treating HCC bone metastasis have yet to be established in clinical practice. Hence, comprehending the mechanisms of HCC bone metastasis is crucial for developing targeted treatment strategies and improving patient prognosis.

Clinically, HCC bone metastasis typically presents as osteolytic bone metastasis, characterized by abnormal bone destruction caused by increased bone absorption mediated by osteoclasts. In clinical practice, the treatment of bone metastasis often involves the use of drugs that inhibit osteoclast bone differentiation/resorption, such as bisphosphonates (BPs) and denosumab. However, the use of these two drugs only provides palliative treatment and does not significantly improve patient prognosis. Moreover, while BPs effectively delay the occurrence of bone metastasis in patients, they also promote the metastasis of tumor cells to visceral organs. Therefore, the identification and characterization of key nodes in HCC bone metastasis, along with the exploration of their molecular mechanisms, will provide crucial scientific foundations for the diagnosis and treatment of HCC bone metastasis.

Ever since Stephen Paget proposed the “seed and soil” hypothesis of tumor metastasis in 1889, there has been mounting evidence confirming that distant metastasis is a coordinated co-evolutionary process between tumor cells and the specific microenvironment of the metastatic organ, with extracellular vesicles playing a crucial role in this process. Extracellular vesicles derived from tumor cells can induce the formation of a specific microenvironment in the metastatic organ to support the occurrence of organ-specific tumor metastasis. Therefore, depending on the tumor type, functional molecules loaded in vesicles can be detected in serum or urine and utilized to predict and diagnose the risk of tumor-specific organ metastasis, assess patient prognosis, and provide opportunities for treatment. However, in the context of HCC bone metastasis, specific vesicle markers for prediction and diagnosis have yet to be identified.

Therefore, the quest for vesicle markers to predict HCC bone metastasis and assess its prognosis is of paramount importance.

### SUMMARY OF THE INVENTION

In view of the above problems, the object of the present invention is to provide a biomarker for diagnosis, treatment, and prognosis for HCC metastasis and application thereof.

The technical content of the present invention is as follows.

The invention provides an application of a biomarker (or a target) for diagnosis, treatment, and prognosis for HCC bone metastasis. The biological gene sequence of VAPA is shown in NM_003574.

The invention provides a biomarker VAPA for diagnosis, treatment, and prognosis for HCC bone metastasis.

The invention further provides a kit for diagnosis, treatment, and prognosis for HCC bone metastasis. The kit includes a reagent for detecting the expression level of the biomarker VAPA.

The kit utilizes either a Western blot or ELISA (enzyme-linked immunosorbent assay) kit.

The invention further provides an application of a reagent or a kit for diagnosis, treatment, and prognosis for HCC bone metastasis.

The invention further provides an application of a reagent, which is configured for detecting the expression level of VAPA in blood and tissue samples, for preparing a reagent or a kit for diagnosis, treatment, and prognosis for HCC bone metastasis.

The advantages of the present inventions is as follows.

The biomarker VAPA of the present invention has the ability to enrich within extracellular large oncosomes (LOs) secreted by HCC cell lines specific to bone metastasis. Its expression is significantly elevated in the serum of HCC patients with specific bone metastasis. The VAPA promotes the differentiation and maturation of osteoclast precursors, thereby facilitating bone metastasis of HCC. It can be used to predict the risk of bone metastasis occurrence in patients, thus serving a preventive role against bone metastasis. In comparison to the diagnostic kits in the prior art that can only detect bone metastasis after it has occurred, the reagent or kit for detecting VAPA expression in this invention offers greater specificity and sensitivity in the early prediction, diagnosis, prognostication, treatment evaluation, drug guidance, and prognosis assessment of HCC bone metastasis. Additionally, the VAPA of this invention may serve as a therapeutic target for bone metastasis.

### BRIEF DESCRIPTION OF DRAWINGS

FIG. 1A shows the enrichment of VAPA in extracellular vesicles LOs secreted by HCC cells with specific bone metastasis, and FIG. 1B shows the high expression of VAPA in the serum of mice experiencing the specific bone metastasis.

FIG. 2 illustrates differential phase-contrast micrographs, TRAP staining images, and corresponding quantitative results of osteoclast precursor cells.

FIG. 3 shows field emission scanning electron microscope (FESEM) images of cytoskeletons, and transmission electron microscope (TEM) images of fused pores in osteoclast precursor cells.

FIG. 4 shows results of bone resorption assay conducted on osteoclast precursor cells cultured on bone slices.

FIG. 5 shows the normalized bioluminescence imaging (BLI) signals of bone metastases and Kaplan-Meier bone metastasis-free survival curve of mice from the indicated experimental group.

FIG. 6 shows representative bone trabecular section images and histological images from the indicated mice in education phase and the experimental metastasis phase.

FIG. 7 illustrates ELISA analysis of serum VAPA levels in healthy donors (health, n=21), HCC patients without bone metastasis (n-BM, n=35), and HCC patients with bone metastasis (BM, n=26).

FIG. 8 shows representative images of VAPA expression in normal liver tissue and various HCC tissues.

FIG. 9 shows Kaplan-Meier analysis of bone metastasis-free survival curves in HCC patients with low versus high expression of VAPA.

### DETAILED DESCRIPTION OF THE INVENTION

The present invention will be described in further detail below through specific implementation examples and accompanying drawings. Various equivalent modifications of the invention are within the meaning of the appended claims.

Unless otherwise specified, all the raw materials and reagents of the present invention are the raw materials and reagents in the conventional market.

### Embodiment 1

A biomarker VAPA for diagnosis, treatment, and prognosis for hepatocellular carcinoma bone metastasis is provided.

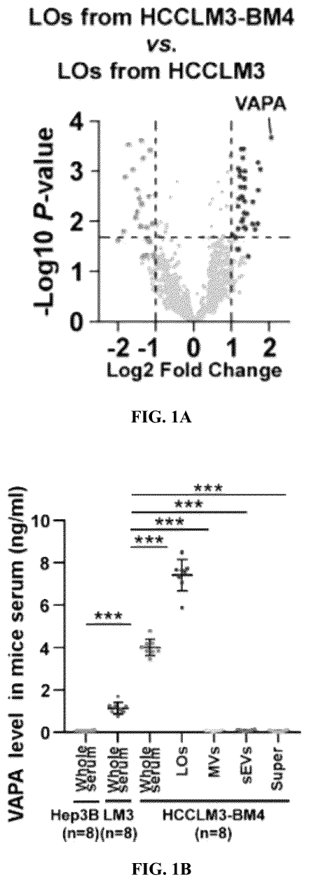

The proteins of LOs, derived from hepatocellular carcinoma cells HCCLM3 and bone metastatic-specific HCCLM3-BM4 cells, were subjected to quantitative mass spectrometry analysis, as shown in FIGS. 1A-1B. FIG. 1A illustrates a volcano plot comparing the LOs of HCCLM3-BM4 cells to those of HCCLM3 cells, highlighting the dysregulated protein expressions. It is evident that VAPA levels were significantly elevated in LOs/HCCLM3-BM4 compared to LOs/HCCLM3, suggesting the potential application of VAPA in the diagnosis of HCC bone metastasis.

FIG. 1B represents the correlation between serum VAPA levels and HCC bone metastasis, as determined by ELISA experiments. Comparing the serum of HCCLM3/mice to that of HCCLM3-BM4/mice, a significant elevation in VAPA levels was observed in the serum of HCCLM3-BM4/mice. This suggests a pre-elevated level of VAPA in the serum prior to HCC bone metastasis. Importantly, other oncosomes isolated from the serum of HCCLM3-BM4/mice, as well as the supernatant, show little detection of the VAPA protein. Conversely, in the isolated LOs from the serum of HCCLM3-BM4/mice, there is a significant increase in the level of VAPA protein. Moreover, the LOs-loaded VAPA level is almost equivalent to the VAPA level detected in the serum. Thus, it is evident that VAPA in the serum holds potential for the diagnosis of HCC bone metastasis.

Th VAPA level in blood samples from HCC patients was measured using the VAPA ELISA kit. The following steps were followed.
(1) Blood samples from HCC patients were collected in serum separation tubes. After clot formation, the tubes were centrifuged at 2000 g for 10 minutes to collect the serum. The samples were diluted 10-fold for analysis, and the undiluted serum was stored at temperatures below −20° C. to avoid repeated freeze-thaw cycles.
(2) The VAPA ELISA kit was used to test the blood samples according to the instructions provided.

Serum samples and all consumables used in the experiment were kept at room temperature for approximately 1 hour.

VAPA levels in both serum and HCC cell culture medium were measured using the VAPA ELISA kit (OKCA01588, Aviva Systems Biology, SanDiego, California, USA), and the analysis was performed following the instructions provided with the kit.

Blood samples from liver cancer patients were collected in serum separation tubes. After clot formation, the tubes were centrifuged at 2000 g for 10 minutes to collect the serum. The samples were diluted 10-fold for analysis, and the undiluted serum was stored at temperatures below −20° C. to avoid repeated freeze-thaw cycles.

The VAPA of the present invention promotes the differentiation and maturation of osteoclast precursors, thereby facilitating HCC bone metastasis. Accordingly, it can be used to predict the risk of bone metastasis in patients, thus serving as a preventive measure against bone metastasis. This is evident from the results and analysis presented in the following figures:

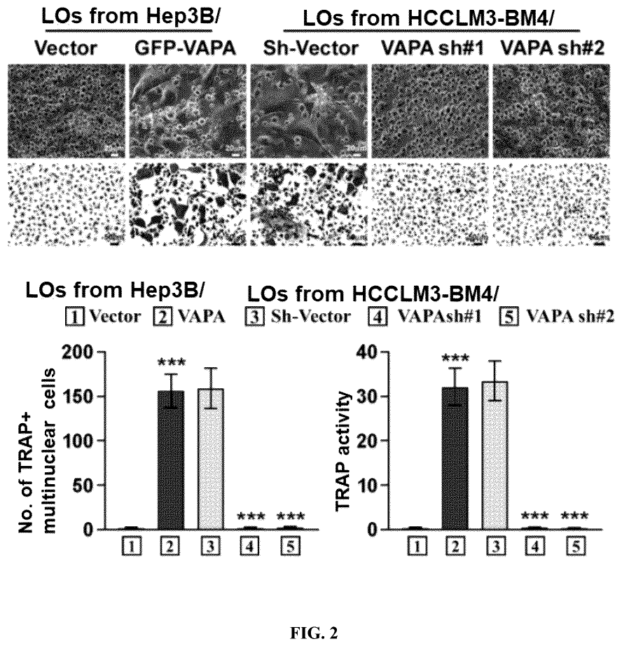

FIG. 2 depicts pre-osteoclasts treated with the indicated LOs, followed by TRAP staining (upper) and quantification (lower) of osteoclasts. Comparing VAPA-overexpressing Hep3B cells-derived LOs to control cells, it is observed that Hep3B cells-derived LOs with high VAPA expression promote osteoclast differentiation, characterized by a significant increase in TRAP⁺ multinuclear cells and an evident rise TRAP activity. Conversely, VAPA-underexpresing HCCLM3-BM4 cells-derived LOs inhibit osteoclast differentiation, indicated by a notable decrease in TRAP⁺ multinuclear cells and a significant reduction in TRAP activity. These results suggest that VAPA plays a crucial role in osteoclast differentiation.

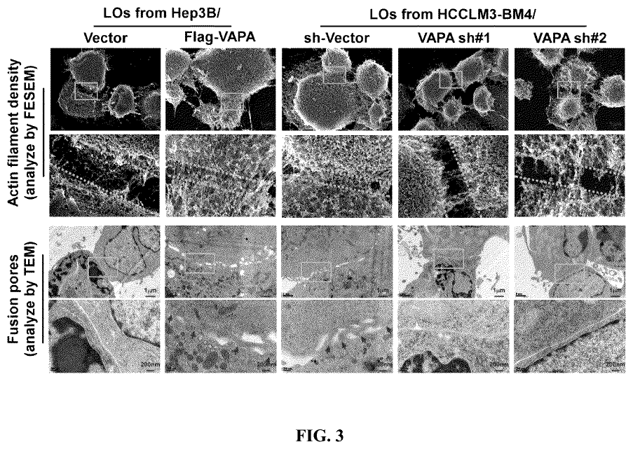

FIG. 3 shows FESEM images of actin filaments in the pre-osteoclasts (upper) and TEM images of fusion pores at the pre-osteoclasts (lower). The results indicate that compared to control cells, LOs derived from VAPA-overexpressing Hep3B cells exhibit increased actin filament density at the surface of two fused pre-osteoclasts, and fusion pores. Conversely, LOs from VAPA-underexpressing HCCLM-BM4 cells shows decreased density at the surface of two fused pre-osteoclasts, and fusion pores. These findings once again demonstrate the significant role of VAPA in osteoclast differentiation.

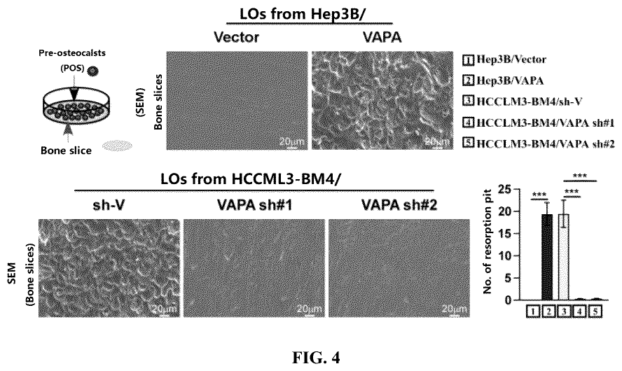

FIG. 4 shows results of bone resorption assays of pre-osteoclasts. SEM was used to observe the bone slices (left), while the number of resorption pits on each bone slice was quantified (right). The results reveal that LOs from VAPA-overexpressing Hep3B cells promote bone resorption by osteoclasts, whereas LOs from VAPA-underexpressing HCCLM3-BM4 inhibit bone resorption. These findings confirm the capacity of VAPA to enhance osteoclast-mediated bone resorption.

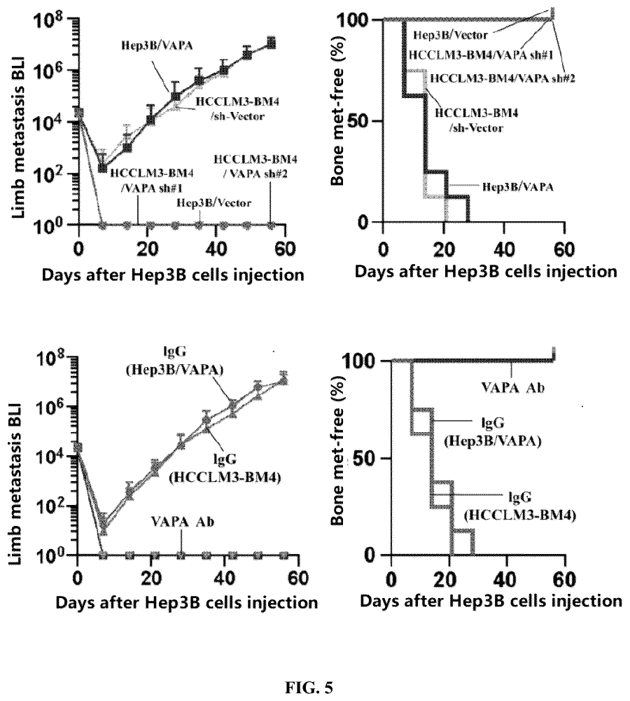

FIG. 5 shows the normalized bioluminescence imaging (BLI) signals of bone metastases and Kaplan-Meier bone metastasis-free survival curve (n=8/group) of mice from the indicated experimental group. LOs derived from tumor cells were injected intraperitoneally into mice and continued for two weeks. Subsequently, HCC cells Hep3B were injected intracardially to examine the metastasis of tumor cells in mice. The results demonstrate that LOs from VAPA-overexpressing Hep3B cells promote bone metastasis of HCC cells, while LOs from VAPA-underexpressing HCCLM3-BM4 cells inhibit bone metastasis of HCC cells. These findings provide evidence of VAPA's role in promoting bone metastasis of HCC.

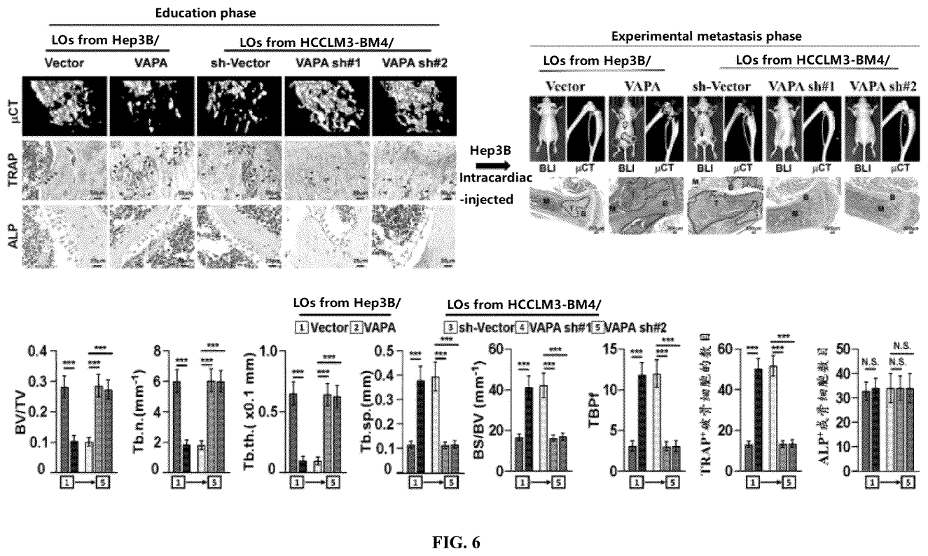

In FIG. 6, upper panel: μCT (bone trabecular section) and histological (TRAP and ALP) images (left) from indicated mice in education phase; BLI and μCT, and histological (H&E) images (right) of bone tumor and lesions from the indicated mice after injecting Hep3B cells in the experimental metastasis phase; lower panel: bone CT parameters, quantification of Trap⁺ osteoclasts and ALP⁺ osteoblasts in bone metastatic lesions.

These results demonstrate that LOs-loaded VAPA promotes osteolytic bone metastasis. Conversely, downregulating VAPA significantly reduces the ability of HCC cells to metastasize to bone and dissolve bone, resulting in a lighter burden of bone metastatic lesions and smaller areas of osteolytic lesions. This indicates that targeting VAPA is a crucial point for inhibiting HCC bone metastasis.

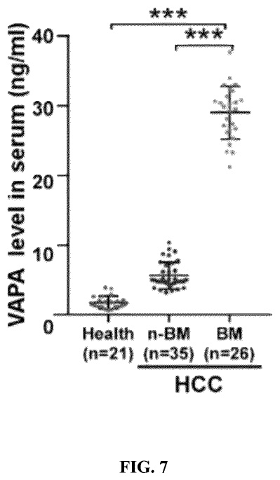

FIG. 7 presents the VAPA levels in the serum of 21 healthy individuals (n=21, Health), 35 HCC patients without bone metastasis (n=35, n-BM), and 26 HCC patients with bone metastasis (n=26, BM), analyzed through ELISA. Compared to healthy individuals and HCC patients without bone metastasis, HCC patients with bone metastasis exhibit a significant elevation in serum VAPA levels.

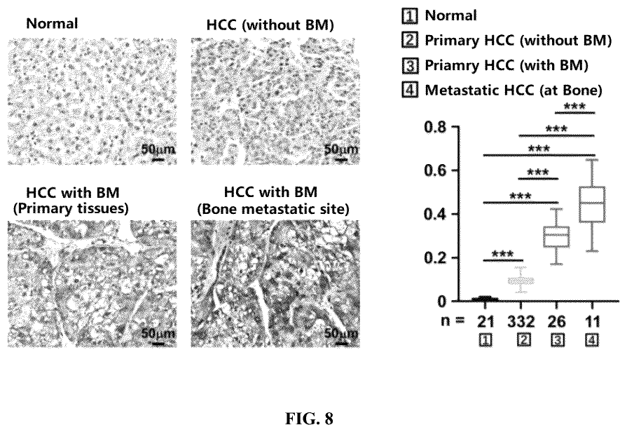

FIG. 8 shows representative images (left) and quantification (right) of VAPA expression in normal liver tissue (n=21), HCC tissues without bone metastasis (n=332), primary HCC tissues with bone metastasis (n=26), and HCC tissues in bone metastatic site (n=11). Scale bar, 50 μm. The HCC tissues from patients was fixed with formalin, embedded in paraffin, and subjected to immunohistochemical staining. The results demonstrate a significant increase in VAPA expression in HCC tissues with bone metastasis, compared to normal liver tissues and HCC tissues without bone metastasis.

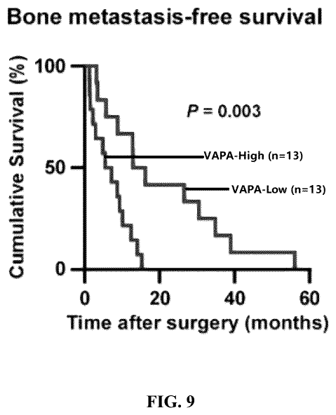

FIG. 9 shows Kaplan-Meier analysis (n=26; P=0.003, log-rank test) of bone metastasis-free survival curves in HCC patients with low versus high expression of VAPA. The results demonstrate that elevated VAPA levels significantly promote bone metastasis in HCC.

Taking into account the aforementioned experimental findings and the accompanying diagrams, this invention signifies the role of VAPA as a biomarker or target for diagnosis, treatment, and prognosis of HCC bone metastasis.

Compared with the diagnostic kits in the prior art that can only detect bone metastasis after it has occurred, the VAPA described in this invention offers a more specific and sensitive assay kit for the early prediction, diagnosis, disease progression assessment, treatment efficacy evaluation, drug guidance, and prognosis assessment of HCC bone metastasis. Additionally, the VAPA may also serve as a target for therapeutic interventions in bone metastasis.

What is claimed is:

1. An application of a reagent that detect VAPA expression levels in blood and tissues for preparing a kit for diagnosis, treatment, and prognosis for hepatocellular carcinoma bone metastasis and application thereof.
